(19) United States
(12) Patent Application Publication
(10) Pub. No.: US 2012/0115941 A1
(43) Pub. Date: May 10, 2012

(54) Compounds Affecting Glycemic Index

(75) Inventors: Dionne Nadine Payn, New South Wales (AU); David Norman Leach, New South Wales (AU); Myrna A. Deseo, New South Wales (AU); Carole Ann Morris, Dublin, CA (US)

(73) Assignee: SACRON INNOVATIONS PTY LIMITED, Toowong, Queensland (AU)

(21) Appl. No.: 13/264,424

(22) PCT Filed: Apr. 16, 2010
(86) PCT No.: PCT/AU2010/000427

§ 371 Date: Jan. 26, 2012

(30) Foreign Application Priority Data
Apr. 17, 2009 (AU) 2009901641

(51) Int. Cl. A61K 31/352; A61P 3/04; A61P 3/10; C07D 311/30

(52) U.S. Cl. 514/456; 549/403

(57) ABSTRACT

Compounds of formula I are disclosed which are useful as glycemic index lowering agents and/or, as α-amylase and/or α-glucosidase inhibitors. Also disclosed are nutritional and/or pharmaceutical compositions and supplements comprising one or more of these compounds. The compounds will be beneficial to patients who require stabilization of their postprandial glucose levels.

### FIELD OF THE INVENTION

This invention relates to compounds which are useful in modulating the glycemic index of a carbohydrate-containing food. More particularly, this invention relates to flavonoids and flavonoid derivatives isolated from sugarcane which are useful as glycemic index lowering agents.

### BACKGROUND OF THE INVENTION

The glycemic index (GI) is a measure of the effect of carbohydrates in the diet on blood glucose levels. Carbohydrates that are broken down quickly during digestion release glucose rapidly into the bloodstream and so have a high GI and conversely those which break down slowly, releasing glucose gradually into the bloodstream, have a low GI.

To determine a food's GI rating, measured portions of the food containing 10-50 grams of carbohydrate are fed to 10 healthy people after an overnight fast. Finger-prick blood samples are taken at 15-30 minute intervals over the next two hours. These blood samples are used to construct a blood sugar response curve for the two hour period. The area under the curve (AUC) is calculated to reflect the total rise in blood glucose levels after eating the test food. The GI rating (%) is calculated by dividing the AUC for the test food by the AUC for the reference food (usually glucose or white bread) and multiplying by 100. A GI value of 55 or less is considered ‘low’, 56-69 is considered ‘medium’ and over 70 is ‘high’.

A lower glycemic index suggests slower rates of digestion and absorption of the foods' carbohydrates and is believed to equate to a lower insulin demand, better long-term blood glucose control and a reduction in blood lipids. It has been shown that individuals who followed a low GI diet over many years were at a significantly lower risk for developing both type 2 diabetes and associated conditions such as cataracts as well as coronary heart disease. High blood glucose levels or repeated glycemic “spikes” following a meal may promote these diseases by both increasing oxidative damage to the vasculature and via the direct increase in insulin levels. Postprandial hyperglycemia has been considered a risk factor mainly associated with diabetes but it is now believed that it also presents an increased risk for atherosclerosis and other conditions in the non-diabetic population.

Low-GI foods, by virtue of their slow digestion and absorption, produce gradual rises in blood sugar and insulin levels and have been shown to improve both glucose and lipid levels in people with diabetes (type 1 and type 2) and have benefits for weight control as they help control appetite and delay hunger. Low GI diets also reduce insulin levels and insulin resistance.

The principal enzymes responsible for the breakdown of carbohydrates in the human body are α-amylase and α-glucosidase and so inhibition of one or both of these enzymes can result in the GI of foods being reduced. Acarbose is an anti-diabetic drug which is a known inhibitor of α-glucosidase. It slows down the digestion of complex carbohydrates and prevents a sharp rise in postprandial glucose levels.

International application PCT/AU2003/001001 describes the use of the flavonoids luteolin, apigenin and tricin in lowering the GI of carbohydrate-containing foods. These compounds displayed varying degrees of moderate activity against both α-amylase and α-glucosidase and the use of tricin demonstrated an ability to reduce postprandial glucose levels.

### SUMMARY OF THE INVENTION

The inventors have identified a need for further compounds which demonstrate efficacy in lowering the glycemic index (GI) of a carbohydrate-containing food.

In a first aspect, although it need not be the only, or indeed the broadest form, the invention resides in a compound of formula I, and/or a salt thereof, for use as a glycemic index lowering agent and/or, as an α-amylase and/or α-glucosidase inhibitor:

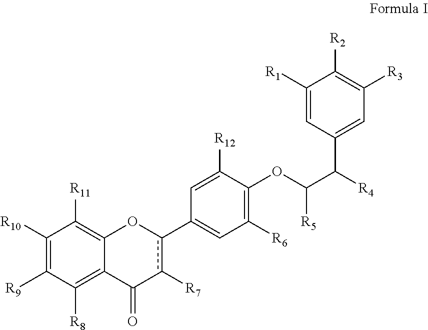

wherein

R₁, R₂, R₃, R₄, R₆, R₇, R₈, R₉, R₁₀, R₁₁ and R₁₂ are independently selected from hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety;

R₅ is hydrogen, CH₂OH, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl, a sugar moiety or R₅ may be represented by the following structure

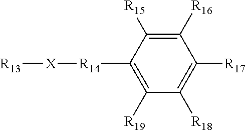

wherein, R₁₃ and R₁₄ are independently selected from alkyl, aryl, alkylene, alkenyl, alkynyl, alkanone, alkanoyl, arylalkyl, arylalkenyl, alkenoyl or carboalkoxy;

X, when present, is oxygen, sulphur, nitrogen, alkyl, alkoxy, alkanoyloxy, alkylene or alkenyl; and

R₁₅, R₁₆, R₁₇, R₁₈ and R₁₉ are independently selected from hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety,

wherein dotted lines may each represent a single bond.

In one embodiment of the first aspect the invention resides in a compound of formula II, and/or a salt thereof, for use as a glycemic index lowering agent and/or, as an α-amylase and/or α-glucosidase inhibitor:

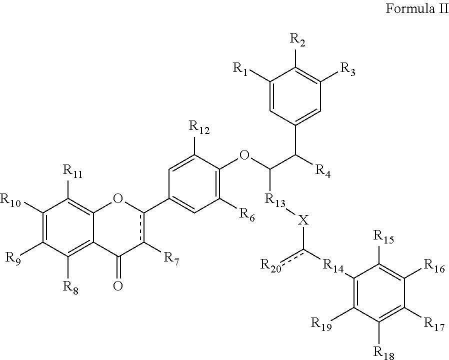

wherein,

R₁, R₂, R₃, R₄, R₆, R₇, R₈, R₉, R₁₀, R₁₁ and R₁₂ are independently selected from hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety;

R₁₃ and R₁₄ are independently selected from alkyl, aryl, alkylene, alkenyl, alkynyl, alkanone, alkanoyl, arylalkyl, arylalkenyl, alkenoyl or carboalkoxy;

X is oxygen, sulphur, nitrogen, alkyl, alkylene or alkenyl;

R₁₅, R₁₆, R₁₇, R₁₈ and R₁₉ are independently selected from hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety;

R₂₀ is selected from hydrogen, oxygen, sulphur, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl or O-alkenoyl; and

wherein dotted lines may each represent a single bond.

In another embodiment of the first aspect the invention resides in a compound of formula III, and/or a salt thereof, for use as a glycemic index lowering agent and/or, as an α-amylase and/or α-glucosidase inhibitor:

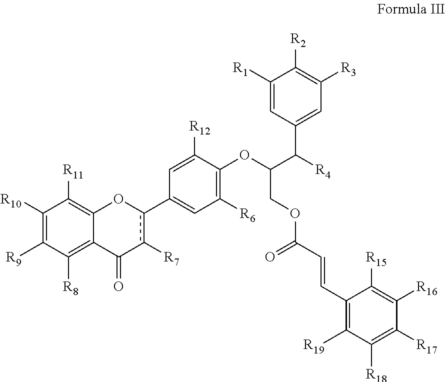

wherein,

R₁, R₂, R₃, R₄, R₆, R₇, R₈, R₉, R₁₀, R₁₁, R₁₂, R₁₅, R₁₆, R₁₇, R₁₈ and R₁₉ are independently selected from hydrogen, hydroxyl, carboxyl, O-alkyl, O-aryl, O-alkenyl, O-alkanoyl or a sugar moiety;

R₁, R₂, R₃, R₄, R₆, R₇, R₈, R₉, R₁₃, R₁₁, R₁₂, R₁₅, R₁₆, R₁₇, R₁₈ and R₁₉ are independently considered in combination with R₁, R₂, R₃, R₄, R₆, R₇, R₈, R_{g}, R₁₀, R₁₁, R₁₂, R₁₅, R₁₆, R₁₇, R₁₈ and R₁₉ as previously defined; and

wherein dotted lines may each represent a single bond.

In yet another embodiment of the first aspect the invention resides in a compound of formula IV, and/or a salt thereof, for use as a glycemic index lowering agent and/or, as an α-amylase and/or α-glucosidase inhibitor:

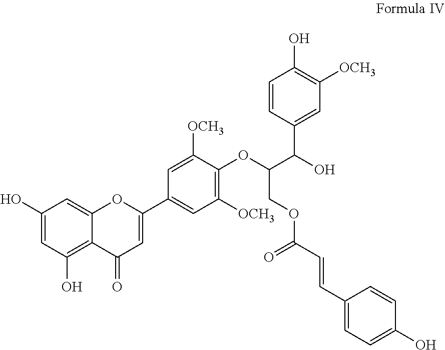

In yet still another embodiment of the first aspect the invention resides in a compound of formula V, and/or a salt thereof, for use as a glycemic index lowering agent and/or, as an α-amylase and/or α-glucosidase inhibitor:

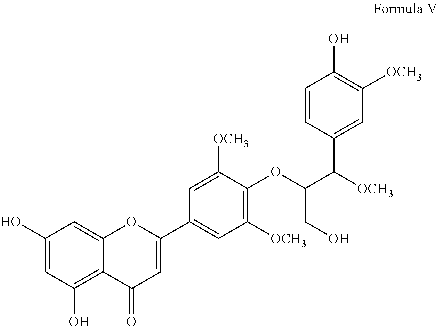

The structures shown for formulae I to V contemplate all stereoisomers.

In one preferred embodiment the compound of the first aspect is selected from the group consisting of tricin-4′-O-[erythro-β-guaiacyl-(9″-O-p-coumaroyl)-glyceryl]ether, tricin-4′-O-[threo-β-guaiacyl-(9″-O-p-coumaroyl)-glyceryl]ether, tricin-4′-O-[threo-β-guaiacyl-(7″-O-methyl)-glyceryl]ether and tricin-4′-O-[erythro-β-guaiacyl-(7″-O-methyl)-glyceryl]ether.

The compounds of formulae I to V may be formulated and/or administered in the form of a pro-drug, for example, with one or more ester moieties.

Preferably, the α-amylase and α-glucosidase are mammalian a- amylase and α-glucosidase.

More preferably, the α-amylase and α-glucosidase are human a- amylase and α-glucosidase.

A second aspect of the invention provides for a compound of formula I and/or a salt thereof, wherein the compound is not tricin-4′-O-[threo-β-guaiacyl-(9″-O-p-coumaroyl)-glyceryl]ether, tricin-4′-O-[erythro-β-guaiacyl-(9″-O-p-coumaroyl)-glyceryl]ether, tricin-4′-O-(erythro-β-guaiacylglyceryl)ether or tricin-4′-O-(threo-β-guaiacylglyceryl)ether.

A third aspect of the invention provides for a compound of formula I and/or a salt thereof, wherein the compound is tricin-4′-O-[threo-β-guaiacyl-(7″-O-methyl)-glyceryl]ether and/or tricin-4′-O-[erythro-β-guaiacyl-(7″-O-methyl)-glyceryl]ether.

A fourth aspect of the invention provides a method of isolating one or more compounds of the first, second or third aspects, including the step of extracting said one or more compounds from a plant, plant part or plant derivative.

In one embodiment the plant is of the family Poaceae otherwise known as Gramineae.

In one embodiment the genus is selected from the group consisting of the genera Saccharum, Erianthus, Miscanthus, Sclerostachya, Narenga, Sasa, Hyparrhenia, Salsola, Avena, Lycopodium and hybrids of these species.

In one embodiment the species is selected from the group consisting of Saccharum officinarum, Saccharum spontaneum, Sasa veitchii (Carr.) Rehder, Hyparrhenia hirta (L.) Stapf, Salsola collina, Avena sativa L. and Lycopodium japonicum.

The parts of the plant may include fruit, seed, bark, leaf, stem, flower, roots and wood.

Preferably, the extract may be obtained from the leaves and/or stem of the plant or from a plant derivative such as a sugarcane processing waste stream, including pre- and post-mill waste streams such as molasses, sugar syrup, field trash, growing tips and mill mud.

A fifth aspect of the invention resides in a compound of the first aspect isolated according to the method of the fourth aspect.

A sixth aspect of the invention resides in a method of treating a disease, disorder or condition responsive to a flavonoid or flavonoid derivative, including the step of administering a compound of the first, second, third and/or fifth aspect.

Suitably, the disease, disorder or condition is responsive to lowering postprandial blood glucose levels and/or, to α-amylase and/or α-glucosidase inhibition.

Preferably, the disease, disorder or condition to be treated is selected from the group consisting of obesity, diabetes and diabetes related conditions such as retinal degeneration, cardiovascular disease, ulcers and kidney failure.

A seventh aspect of the invention provides a nutritional composition comprising a compound of the first, second, third and/or fifth aspect, or a pharmaceutically acceptable salt thereof, and a nutritional component.

The compound of the first aspect may be selected from the group consisting of tricin-4′-O-[erythro-β-guaiacyl-(9″-O-p-coumaroyl)-glyceryl]ether, tricin-4′-O-[threo-β-guaiacyl-(9″-O-p-coumaroyl)-glyceryl]ether, tricin-4′-O-[threo-β-guaiacyl-(7″-O-methyl)-glyceryl]ether and tricin-4′-O-[erythro-β-guaiacyl-(7″-O-methyl)-glyceryl]ether.

The nutritional composition may further comprise a food additive.

Preferably, the food additive is selected from the group consisting of molasses, poly phenols, kidney bean and kidney bean extracts including phaseolamin, a fibre additive and an acid.

The nutritional component is a carbohydrate-containing food.

An eighth aspect of the invention provides a pharmaceutical composition for the treatment or prophylaxis of a disease, disorder or condition comprising an effective amount of a compound of the first, second, third and/or fifth aspect, or a pharmaceutically acceptable salt thereof, and a pharmaceutically acceptable carrier, diluent and/or excipient.

The pharmaceutical composition may include more than one compound of the first, second, third and/or fifth aspect.

The more than one compound may be in any ratio.

The one or more compounds of the first aspect may be selected from the group consisting of tricin-4′-O-[erythro-β-guaiacyl-(9″-O-p-coumaroyl)-glyceryl]ether, tricin-4′-O-[threo-β-guaiacyl-(9″-O-p-coumaroyl)-glyceryl]ether, tricin-4′-O-[threo-β-guaiacyl-(7″-O-methyl)-glyceryl]ether and tricin-4′-O-[erythro-β-guaiacyl-(7″-O-methyl)-glyceryl]ether.

A ninth aspect of the invention provides a nutritional supplement comprising an effective amount of a compound of the first, second, third and/or fifth aspect, or a pharmaceutically acceptable salt thereof, and an additive.

The nutritional supplement may be prepared in an ingestible solid or liquid form including capsules, tablets, powders, pills, solutions, drinks or granules.

The additive may be selected from the group consisting of fillers, binders, humectants, excipients, processing aides, vitamins and minerals.

A tenth aspect of the invention provides for the use of a compound of the first, second, third and/or fifth aspect, or a pharmaceutically acceptable salt thereof, in the manufacture of a medicament for the treatment or prophylaxis of a disease, disorder or condition.

The various features and embodiments of the present invention, referred to in individual sections above apply, as appropriate, to other sections, mutatis mutandis. Consequently features specified in one section may be combined with features specified in other sections as appropriate.

In this specification, the terms “comprises”, “comprising” or similar terms are intended to mean a non-exclusive inclusion, such that a product, composition, method, system or apparatus that comprises a list of elements does not include those elements solely, but may well include other elements not listed.

### BRIEF DESCRIPTION OF DRAWINGS

FIG. 1 shows the structure of certain compounds isolated from a methanolic sugarcane leaf extract along with a number of control compounds;

FIG. 2 is a schematic representation of the isolation of certain compounds, including compounds 5 to 8, from the residue of a methanolic extract of Saccharum officinarum leaves; and

FIG. 3 is a representation of significant long-range heteronuclear multiple bond correlations for compounds 7 and 8.

### DETAILED DESCRIPTION

The present invention arises from the discovery of flavonoid derivatives which demonstrate surprisingly high levels of efficacy as inhibitors of α-amylase and/or α-glucosidase enzymes. These compounds are suitable for use as glycemic index (GI) lowering agents to provide control over blood glucose levels.

In a first aspect, although it need not be the only, or indeed the broadest form, the invention resides in a compound of formula I, and/or a salt thereof, as hereinbefore described, for use as a glycemic index lowering agent and/or, as an α-amylase and/or α-glucosidase inhibitor:

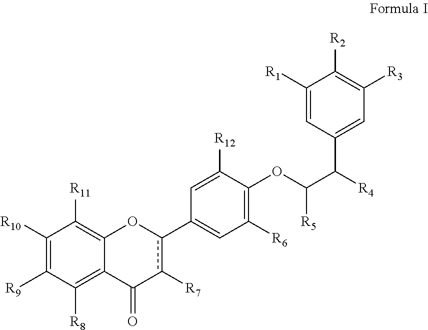

wherein,

R₁, R₂, R₃, R₄, R₆, R₇, R₈, R₉, R₁₀, R₁₁ and R₁₂ are independently selected from hydrogen, alkyl, alkenyl, arylalkyl, hydroxyalkyl, hydroxyl; aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety;

R₅ is hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl, a sugar moiety or R₅ may be represented by the following structure

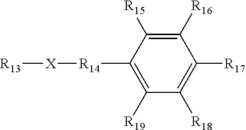

wherein, R₁₃ and R₁₄ are independently selected from alkyl, aryl, alkylene, alkenyl, alkynyl, alkanone, alkanoyl, arylalkyl, arylalkenyl, alkenoyl or carboalkoxy;

X, when present, is oxygen, sulphur, nitrogen, alkyl, alkoxy, alkanoyloxy, alkylene or alkenyl;

R₁₅, R₁₆, R₁₇, R₁₈ and R₁₉ are independently selected from hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety, and

wherein dotted lines may each represent a single bond.

The term “glycemic index lowering agent” as used herein, refers to a compound which, upon appropriate administration in conjunction with a carbohydrate-containing food, is capable of reducing the postprandial blood glucose level in a subject compared to that level obtained after administration of the food alone.

The term “pharmaceutically acceptable salt”, as used herein, refers to salts which are toxicologically safe for systemic or localised administration such as salts prepared from pharmaceutically acceptable non-toxic bases or acids including inorganic or organic bases and inorganic or organic acids. The pharmaceutically acceptable salts may be selected from the group including alkali and alkali earth, ammonium, aluminium, iron, amine, glucosamine, chloride, sulphate, sulphonate, bisulphate, nitrate, citrate, tartrate, bitarate, phosphate, carbonate, bicarbonate, malate, maleate, napsylate, fumarate, succinate, acetate, benzoate, terephthalate, palmoate, piperazine, pectinate and S-methyl methionine salts and the like.

The term “alkyl” refers to optionally substituted linear and branched hydrocarbon groups having 1 to 20 carbon atoms. Where appropriate, the alkyl group may have a specified number of carbon atoms, for example, C₁-C₆ alkyl which includes alkyl groups having 1, 2, 3, 4, 5 or 6 carbon atoms in linear or branched arrangements. Non-limiting examples of alkyl groups include methyl, ethyl, propyl, isopropyl, butyl, s- and t-butyl, pentyl, 2-methylbutyl, 3-methylbutyl, hexyl, heptyl, 2-methylpentyl, 3-methylpentyl, 4-methylpentyl, 2-ethylbutyl, 3-ethylbutyl, octyl, nonyl, decyl, undecyl, dodecyl, tridecyl, tetradecyl, pentadecyl.

The term “alkylene” refers to a saturated aliphatic chain substituted at either end, also known as an alkanediyl. Non-limiting examples may include —CH₂—, —CH₂CH₂— and —CH₂CH₂CH₂—.

The term “alkenyl” refers to optionally substituted unsaturated linear or branched hydrocarbon groups, having 2 to 20 carbon atoms and having at least one carbon-carbon double bond. Where appropriate, the alkenyl group may have a specified number of carbon atoms, for example, C₂-C₆ alkenyl which includes alkenyl groups having 2, 3, 4, 5 or 6 carbon atoms in linear or branched arrangements. Non-limiting examples of alkenyl groups include, ethenyl, propenyl, isopropenyl, butenyl, s- and t-butenyl, pentenyl, hexenyl, hept-1,3-diene, hex-1,3-diene, non-1,3,5-triene and the like.

The term “alkynyl” refers to optionally substituted unsaturated linear or branched hydrocarbon groups, having 2 to 20 carbon atoms and having at least one carbon-carbon triple bond. Where appropriate, the alkynyl group may have a specified number of carbon atoms, for example, C₂-C₆ alkynyl groups have 2, 3, 4, 5 or 6 carbon atoms in linear or branched arrangements. Non-limiting examples of alkynyl groups include ethynyl, propynyl, butynyl, penrynyl, hexynyl and the like.

“Aryl” means a C₆-C₁₄ membered monocyclic, bicyclic or tricyclic carbocyclic ring system having up to 7 atoms in each ring, wherein at least one ring is aromatic. Examples of aryl groups include, but are not limited to, phenyl, naphthyl, tetrahydronaphthyl, indanyl and biphenyl. The aryl may comprise 1-3 benzene rings. If two or more aromatic rings are present, then the rings may be fused together, so that adjacent rings share a common bond.

“Alkanoyl” means an acyl moiety of a straight or branched configuration having 1-20 carbon atoms. Examples of alkanoyl groups include, but are not limited to, acetyl, propionoyl, butyryl, isobutyryl, pentanoyl and hexanoyl.

“Alkenoyl” means alkenylcarbonyl in which alkenyl is as defined above. Examples of alkenoyl groups include, but are not limited to, pentenoyl, hexenoyl or heptenoyl.

The term “carboalkoxy” refers to an alkyl ester of a carboxylic acid, wherein alkyl has the same definition as found above. Examples include carbomethoxy, carboethoxy, carboisopropoxy and the like.

The term “arylalkyl” defines an alkylene, such as —CH₂— for example, which is substituted with an aryl group that can be substituted or unsubstituted as defined above. Examples of an “arylalkyl” include benzyl, phenethylene and the like.

“Alkanone” refers to a ketone substituent with 2 to 12 carbon atoms in a linear, branched or cyclic arrangement, optionally substituted with 1 to 5 substituents independently selected at each occurrence from halogens, cyano or nitro.

The term “hydroxyalkyl” refers to an aliphatic group, which may be branched, having from 1 to 12 carbon atoms, and further comprising at least one hydroxyl group on the main carbon chain and/or on a side chain. Hydroxyalkyl groups include, by way of example only, CH₂OH, 2-hydroxy-1,1-dimethyl-ethyl, 1-hydroxymethyl-2-methyl-propyl and 2-hydroxy-propyl.

In one preferred embodiment of the first aspect the compound is a compound of Formula IV, and/or a salt thereof.

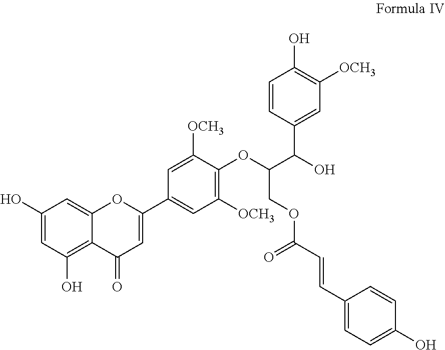

In another preferred embodiment of the first aspect the compound is a compound of Formula V, and/or a salt thereof.

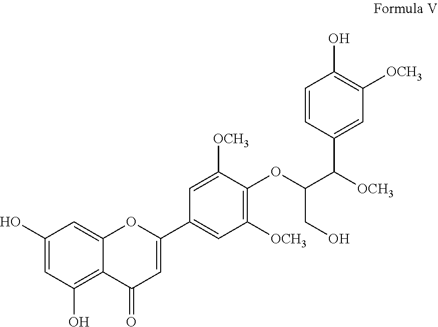

In one embodiment the compound of the first aspect is tricin-4′-O-[erythro-β-guaiacyl-(9″-O-p-coumaroyl)-glyceryl]ether. In a further embodiment the compound of the first aspect is tricin-4′-O-[threo-β-guaiacyl-(9″-O-p-coumaroyl)-glyceryl]ether. In yet a further embodiment the compound of the first aspect is tricin-4′-O-[threo-β-guaiacyl-(7″-O-methyl)-glyceryl]ether. In still yet a further embodiment the compound of the first aspect is tricin-4′-O-[erythro-β-guaiacyl-(7″-O-methyl)-glyceryl]ether The activity data of these compounds against α-amylase and α-glucosidase enzymes are shown in table 1 where they are labelled as compounds 7 (erythro p-coumaroyl form), 8 (threo p-coumaroyl form), 5 (threo O-methyl form) and 6 (erythro O-methyl form).

This invention provides compounds, or salts, solvates or stereoisomers thereof, as glycemic index lowering agents and/or α-amylase and/or α-glucosidase inhibitors. These compounds may contain one or more chiral or asymmetric centres and, when such a chiral centre or centres are present, this invention may be directed to racemic mixtures, pure stereoisomers (i.e. individual enantiomers or diastereomers) and stereoisomer-enriched mixtures of such isomers, unless otherwise indicated. When a particular stereoisomer is shown, it will be understood by those skilled in the art that minor amounts of other stereoisomers may be present in the compositions of this invention, unless otherwise indicated, provided that the utility of the composition as a whole is not eliminated by the presence of such other stereoisomers.

The invention thus includes compounds in substantially pure isomeric form at one or more asymmetric centres e.g., greater than about 90% ee, such as about 95% or 97% ee, or greater than 99% ee, as well as mixtures thereof. Such isomers may be obtained by isolation from natural sources, by asymmetric synthesis, for example using chiral intermediates, or by chiral resolution.

Additionally, where applicable, all cis/trans or E/Z isomers (geometric isomers), erythro and threo forms, tautomeric forms and topoisomeric forms of the compounds of the first aspect are included within the scope of this invention, unless otherwise specified.

Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., University of Notre Dame Press, Notre Dame, Ind. 1972), the entire disclosures of which are herein incorporated by reference.

The absolute stereochemistry of stereoisomers may be determined by methods which are well known in the art such as x-ray crystallography of crystalline products or crystalline intermediates which are derivatised, if necessary, with a reagent containing an asymmetric centre of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallisation or chromatography. The coupling reaction is often the formation of salts using an enantiomerically pure acid or base. The diastereomeric derivatives may then be converted to the pure enantiomers by cleavage of the added chiral residue. The racemic mixture of the compounds can also be separated directly by chromatographic methods utilising chiral stationary phases, which methods are well known in the art.

Alternatively, any enantiomer of a compound may be obtained by stereoselective synthesis using optically pure starting materials or reagents of known configuration by methods well known in the art.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centres of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refers to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New -York; and Eliel, E. and Wilen, S. Stereochemistry of Organic Compounds (1994) John Wiley & Sons, Inc., New York.

The terms “racemic mixture” and “racemate” refer to an equimolar mixture of two enantiomeric species, devoid of optical activity.

Two common prefixes used to designate the relative configuration of an acyclic structure or partial structure having adjacent stereogenic centres are “threo” and “erythro”. When a molecule is drawn in Fischer projection form the erythro isomer has two identical substituents on the same side and the threo isomer has them on opposite sites.

The compounds of the first aspect can reduce postprandial hyperglycemia and will therefore be useful in the treatment of any condition responsive to lowering postprandial blood glucose levels and/or, to α-amylase and/or α-glucosidase inhibition.

The disease, disorder or condition to be treated may be selected from a large number of conditions, some non-limiting examples of which are obesity, diabetes and numerous diabetes related conditions including retinal degeneration, cardiovascular disease, ulcers and kidney failure. Other diabetes related conditions are well known in the literature.

The compound may be administered simultaneously with the carbohydrate-containing meal which is being ingested. Alternatively, the compound may be administered prior to ingestion of the carbohydrate-containing meal. The compound may also be administered subsequent to the ingestion of the carbohydrate-containing meal but still within such a time frame that it is able to have the desired GI lowering effect i.e. before substantially complete digestion of the carbohydrates.

The compound of the first aspect may be an inhibitor of α-amylase and/or α-glucosidase.

These enzymes are acknowledged as being the principal enzymes involved in carbohydrate digestion.

Preferably, the α-amylase and α-glucosidase are mammalian α-amylase and α-glucosidase.

More preferably, the α-amylase and α-glucosidase are human α-amylase and α-glucosidase.

Human α-amylase and α-glucosidase may comprise more than one isoform in which case at least one isoform will be inhibited by a compound of the present invention.

Table 1 indicates the inhibitory activity of compounds 5 to 8 which were isolated from a methanolic sugarcane extract, along with a number of controls, against porcine α-amylase, bakers yeast α-glucosidase and rat intestinal α-glucosidase. The column header “CMP” represents compound number. Compounds 5 to 8 were those compounds of the invention isolated from a methanolic sugarcane extract while compounds 18 and 19 (Apigenin and Luteolin), as well as acarbose and fucoidan, were purchased controls. Acarbose is an anti-diabetic drug which is known to strongly inhibit the α-glucosidase enzyme while fucoidan is an inhibitor of yeast α-glucosidase. Compound 4 (Tricin) is a known and commercially important glycemic index lowering compound isolated from sugarcane leaf and sugarcane mill processing waste stream.

It is apparent from table 1 that compounds 7 and 8, being tricin-4′-O-[erythro-β-guaiacyl-(9″-O-p-coumaroyl)-glyceryl]ether and tricin-4′-O-[threo-β-guaiacyl-(9″-O-p-coumaroyl)-glyceryl]ether, respectively, demonstrate surprisingly high levels of efficacy in comparison to known GI lowering agents such as tricin, luteolin and apigenin. Particularly, compounds 7 and 8 show levels of inhibition of α-amylase which are between 50 to 110 fold greater than those for tricin (4).

The compounds of the invention may display activity against α-amylase and/or α-glucosidase enzymes which is at least 3 times that observed for tricin and which activity may be at least 4, 5, 6, 7, 8, 9, 10, 15, 20, 30, 40, 50, 60, 70, 80, 90, 100, 110, 120, 130, 140 or 150 times greater than the activity observed for tricin.

Compound 7 displayed an IC₅₀ value of 2.0 μM against porcine α-amylase and also demonstrated a higher percentage inhibition at 200 μM against rat intestinal α-glucosidase than tricin. This compound is, therefore, a suitable GI lowering agent as it exhibits excellent levels of inhibition against both of the principal enzymes involved in carbohydrate digestion.

Compound 8 showed even greater efficacy than compound 7 against porcine α-amylase and with only a slightly lower level of activity against rat intestinal α-glucosidase than tricin. This compound represents a valuable GI lowering agent as it exhibits levels of activity against porcine α-amylase almost 110-fold greater than tricin and 130-fold greater than acarbose as well as a notable level of inhibition of rat intestinal α-glucosidase.

As demonstrated with compounds 7 and 8, it is envisaged that the compounds of the first aspect may well demonstrate different degrees of efficacy against the α-amylase and α-glucosidase enzymes. So long as the compound is effective in inhibiting at least one of these enzymes then it can be useful as a GI lowering agent.

Although not wishing to be bound by any particular theory it is postulated that the potency of compounds 7 and 8 is a result of three aromatic moieties (the flavonoid core A-ring, the guaiacylglyceryl group and the p-coumaroyl group) comprising free hydroxyl groups binding different amino acids within the enzyme binding pocket.

Novel compounds 5 and 6 also display strong levels of activity against one or more of the enzymes tested, in particular compounds 5 and 6 demonstrated significantly improved activity, i.e. a 3 to 4 fold increase, against porcine α-amylase compared with tricin. Although not quite as efficacious as compounds 7 and 8, these two compounds will also be useful either alone or in combination with other compounds as glycemic index lowering agents and/or, as α-amylase and/or α-glucosidase inhibitors.

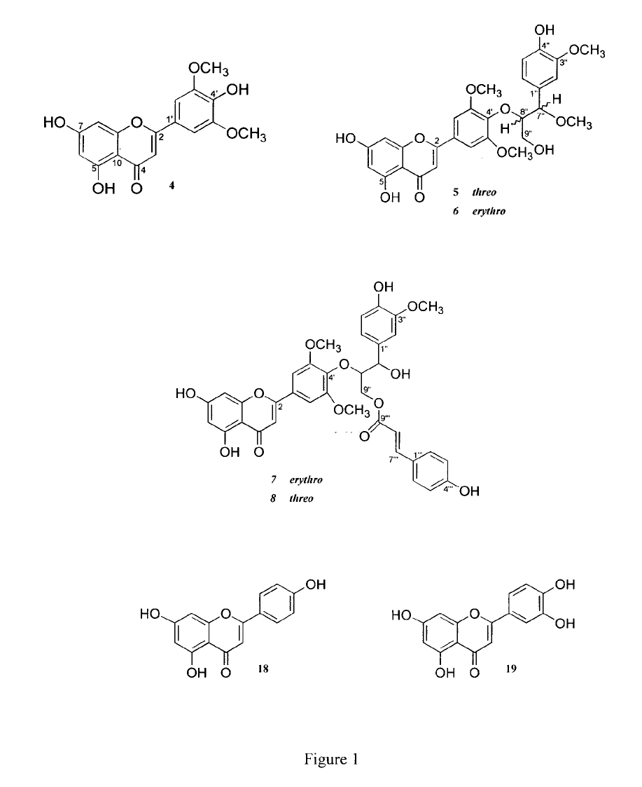

The structures of compounds 5 to 8, which were isolated from a methanolic sugarcane extract, are shown in FIG. 1. Compounds 5 to 8 are flavonoid derivatives, known as flavonolignans, which consist of the threo and erythro diastereomers of two different stereoisomeric compounds. As previously discussed, compounds 7 and 8 displaying the p-coumaroyl group demonstrate the greatest efficacy against both the α-amylase and α-glucosidase enzymes.

A second aspect of the invention provides for a compound of formula I and/or a salt thereof, wherein the compound is not tricin-4′-O-[threo-β-guaiacyl-(9″-O-p-coumaroyl)-glyceryl]ether, tricin-4′-O-[erythro-β-guaiacyl-(9″-O-p-coumaroyl)-glyceryl]ether, tricin-4′-O-(erythro-β-guaiacylglyceryl)ether or tricin-4′-O-(threo-β-guaiacylglyceryl)ether.

In a third aspect the invention provides for a compound of formula I, and/or a salt thereof, wherein the compound is tricin-4′-O-[threo-β-guaiacyl-(7″-O-methyl)-glyceryl]ether (compound 5 in table 1) and/or tricin-4′-O-[erythro-β-guaiacyl-(7″-O-methyl)-glyceryl]ether (compound 6 in table 1). These compounds of the invention are novel compounds.

The compounds of the present invention may be obtained by isolation from a plant, plant part, terrestrial organism, terrestrial organism part, marine organism and/or marine organism part, or by derivatisation of the isolated compound, or by derivatisation of a related compound or by synthesis. The synthesis may be total or semi-synthesis. Preferably, the compounds are obtained by isolation from a plant or plant part.

A fourth aspect of the invention provides a method of isolating one or more compounds of the first, second or third aspects, including the step of extracting said one or more compounds from a plant, plant part or plant derivative.

In one embodiment the plant is of the family Poaceae, otherwise known as Gramineae.

In a further embodiment the plant genus is selected from the group consisting of the genera Saccharum, Erianthus, Miscanthus, Sclerostachya, Narenga, Sasa, Hyparrhenia, Salsola, Avena, Lycopodium and hybrids of these species.

In another embodiment the plant species is selected from the group consisting of Saccharum officinarum, Saccharum spontaneum, Sasa veitchii (Carr.) Rehder, Hyparrhenia hirta (L.) Stapf, Salsola collina, Avena sativa L. and Lycopodium japonicum.

The parts of the plant may include fruit, seed, bark, leaf, stem, flower, roots and wood.

Preferably, the extract may be obtained from the leaves and/or stem of the plant or from one or more plant derivatives such as sugarcane processing waste streams, including pre- and post-mill waste streams, such as molasses, sugar syrup, field trash, growing tips and mill mud.

When the extract is obtained from the leaves of the sugarcane plant the biomass may be subjected to an initial solvent extraction, for example with a solvent such as, but not limited to, methanol and/or dichloromethane (DCM). The extraction may then be subjected to separation by, for example, silica flash column or reverse-phase separation methods. The fractions may then be further separated by preparative high performance liquid chromatography (HPLC) and may be analysed by analytical HPLC and pooled according to the retention time of compounds found in the samples. Further details of the isolation method are discussed in the examples.

Other compounds of the invention may be obtained by derivatising compounds of the first aspect isolated from the plants or parts of plants as outlined above.

Derivatives of the natural compounds can be obtained by techniques known in the art. For example, hydroxy groups may be oxidised, to ketones, aldehydes or carboxylic acids by exposure to oxidising agents such as chromic acid, Jones' reagent, potassium permanganate (KMnO₄), peracids such as metachloroperbenzoic acid (mCPBA) or dioxiranes such as dimethyldioxirane (DMDO) and methyl(trifluoromethyl)dioxirane (TFDO). Oxidising agents may be chosen such that other functional groups in the molecule are, or are not, also oxidised. For example, a primary alcohol may be selectively oxidised to an aldehyde or carboxylic acid in the presence of secondary alcohols using reagents such as RuCl₂(PPh₃)₃-benzene. Secondary alcohols may be selectively oxidised to ketones in the presence of a primary alcohol using Cl₂-pyridine or NaBrO₃-ceric-ammonium nitrate. Alcohols may be oxidised in the presence of double and triple bonds and without epimerisation at adjacent stereocentres using Jone's reagent. Alternatively, reagents chosen may be less selective resulting in oxidation at more than one functional group.

Hydroxy groups may be derivatised by, for example, etherification or acylation. For example, ethers may be prepared by formation of an alkoxide ion in the presence of base and reacting the alkoxide with an appropriate alkylhalide, alkenylhalide, alkynylhalide or arylhalide. Similarly acylation may be achieved by formation of an alkoxide ion and reaction with an appropriate carboxylic acid or activated carboxylic acid (such as an anhydride).

Acyl groups may be hydrolysed to provide alcohols by acid or base hydrolysis as is known in the art.

Silyl groups may be introduced onto hydroxy groups to provide silyl ethers using mild base and a silyl chloride reagent, for example Me₃SiCl and triethylamine in tetrahydrofuran (THF) or agents such as MeSiNHCO₂SiMe₃ in THF.

Sulfonates may be readily introduced onto hydroxy groups by reaction with a suitable sulfonate group. For example, methanesulfonates may be introduced by treatment of a hydroxy group with mesyl chloride (MsCl) and triethylamine in dichloromethane. Tosylate groups may be introduced by reaction of a hydroxy group with tosyl chloride (TsCl) and pyridine. Allylsulfonates may be introduced by reaction of a hydroxy group with allylsulfonyl chloride and pyridine in dichloromethane.

Ketones may be reduced to secondary alcohols by reducing agents such as lithium aluminium hydride and other metal hydrides without reducing double bonds, including a-unsaturated ketones.

Double bonds and triple bonds may be reduced to single bonds using catalytic reduction, for example, H₂/Pd. Double bonds may also be oxidised to epoxides using oxidising agents such as per acids, for example meta-Chloroperoxybenzoic acid (mCPBA) or dioxiranes, such as Dimethyldioxirane (DMDO). Double bonds may also be subject to addition reactions to introduce substituents such as halo groups, hydroxy or alkoxy groups and amines.

A person skilled in the art is be able to determine suitable conditions for obtaining derivatives of isolated compounds, for example, by reference to texts relating to-synthetic methodology, non-limiting examples of which are Smith M. B. and March J., March's Advanced Organic Chemistry, Fifth Edition, John Wiley & Sons Inc., 2001 and Larock R. C., Comprehensive Organic Transformations, VCH Publishers Ltd., 1989. Furthermore, selective manipulations of functional groups may require protection of other functional groups. Suitable protecting groups to prevent unwanted side reactions are provided in Green and Wuts, Protective Groups in Organic Synthesis, John Wiley & Sons Inc., 3rd Edition, 1999.

The compounds of the invention may also be synthesised from commercially available starting materials.

A fifth aspect of the invention resides in a compound of the first, second or third aspect isolated according to the method of the fourth aspect.

In a sixth aspect the invention resides in a method of treating a disease, disorder or condition responsive to a flavonoid or flavonoid derivative, including the step of administering an effective amount of a compound of the first, second, third and/or fifth aspect.

The disease, disorder or condition to be treated will be caused by, exacerbated by or in some way related to the effects of postprandial hyperglycemia and will be responsive to lowering postprandial blood glucose levels and/or, to α-amylase and/or α-glucosidase inhibition.

Preferably, the disease, disorder or condition to be treated is selected from the group consisting of obesity, coronary heart disease, diabetes and diabetes related conditions such as retinal degeneration, cardiovascular disease, ulcers and kidney failure.

In a seventh aspect the invention provides a nutritional composition comprising a compound of the first, second, third and/or fifth aspect, or a pharmaceutically acceptable salt thereof, and a nutritional component.

The nutritional composition may further comprise one or more food additives to aid in lowering the GI of the meal, for example, a fibre additive which slows digestion or an acid, such as vinegar or lemon juice, which slows down the rate at which the stomach empties.

Preferably, the food additive is selected from the group consisting of molasses; poly-phenols, kidney bean and kidney bean extracts including phaseolamine, a fibre additive and an acid.

The food additive may also comprise recognised health supplements such as vitamins, amino acid supplements, digestive supplements and the like.

The nutritional composition may include inactive or pro-drug forms of the compounds of the first aspect which are subsequently activated or converted to their active form after ingestion.

The compound of the first aspect may be provided in substantially pure form or as part of a sugarcane leaf extract containing other, potentially, beneficial compounds.

The nutritional component will comprise a carbohydrate-containing food. Preferably, the nutritional component will comprise a carbohydrate-containing food which has a medium to high GI which it would be desirable to lower, for example, white bread, white rice, potatoes and high sugar-content breakfast cereals.

Preferably, the compound of the first, second, third and/or fifth aspect in the nutritional composition is selected from the group consisting of tricin-4′-O-[erythro-β-guaiacyl-(9″-O-p-coumaroyl)-glyceryl]ether (compound 7), tricin-4′-O-[threo-β-guaiacyl-(9″-O-p-coumaroyl)-glyceryl]ether (compound 8), tricin-4′-O-[threo-β-guaiacyl-(7″-O-methyl)glyceryl]ether (compound 5) and tricin-4′-O-[erythro-β-guaiacyl-(7″-O-methyl)-glyceryl]ether (compound 6).

In an eighth aspect the invention provides a pharmaceutical composition for the treatment or prophylaxis of a disease, disorder or condition comprising an effective amount of a compound of the first, second, third and/or fifth aspect, or a pharmaceutically acceptable salt thereof, and a pharmaceutically acceptable carrier, diluent and/or excipient.

The pharmaceutical composition may include more than one compound of the first, second, third and/or fifth aspect. The one or more compounds of the first, second, third and/or fifth aspect may be 1, 2, 3, 4, 5, 6, 7, 8, 9 or 10 compounds. When the composition includes more than one compound then the compounds may be in any ratio.

Preferably, the compound of the first, second, third and/or fifth aspect in the pharmaceutical composition is selected from the group consisting of tricin-4′-O[erythro-β-guaiacyl-(9″-O-p-coumaroyl)-glyceryl]ether (compound 7), tricin-4′-O-[threo-β-guaiacyl-(9″-O-p-coumaroyl)-glyceryl]ether (compound 8), tricin-4′-O-[threo-β-guaiacyl-(7″-O-methyl)-glyceryl]ether (compound 5) and tricin-4′-O-[erythro-β-guaiacyl-(7″-O-methyl)-glyceryl]ether (compound 6).

The compounds of the first, second, third and/or fifth aspect are present in an amount sufficient to prevent, inhibit or ameliorate the diseases, disorders or conditions which are the subject of treatment. Suitable dosage forms and rates of the compounds of the first aspect and the pharmaceutical compositions containing such may be readily determined by those skilled in the art.

The disease, disorder or condition to be treated will be caused by, exacerbated or in some way related to the effects of postprandial hyperglycemia and will be responsive to lowering postprandial blood glucose levels and/or, to α-amylase and/or α-glucosidase inhibition.

Preferably, the disease, disorder or condition to be treated is selected from the group consisting of obesity, coronary heart disease, diabetes and diabetes related conditions such as retinal degeneration, cardiovascular disease, ulcers and kidney failure.

Dosage forms include tablets, dispersions, suspensions, injections, solutions, syrups, troches, capsules, suppositories, aerosols, transdermal patches and the like. These dosage forms may also include injecting or implanting devices designed specifically for, or modified to, controlled release of the pharmaceutical composition.

Controlled release of the therapeutic agent may be achieved by coating the same, for example, with hydrophobic polymers including acrylic resins, waxes, higher aliphatic alcohols, polylactic and polyglycolic acids and certain cellulose derivates such as hydroxypropylmethyl cellulose. In addition, the controlled release may be affected by using other polymer matrices, liposomes and/or microspheres.

Suitably, the pharmaceutical composition comprises a pharmaceutically acceptable excipient or an acceptable excipient. By “pharmaceutically acceptable excipient” is meant a solid or liquid filler, diluent or encapsulating substance that may be safely used in systemic administration. Depending upon the particular route of administration, a variety of carriers, well known in the art may be used. These carriers or excipients may be selected from a group including sugars, starches, cellulose and its derivates, malt, gelatine, talc, calcium sulphate, vegetable oils, synthetic oils, polyols, alginic acid, phosphate buffered solutions, emulsifiers, isotonic saline, and pyrogen-free water.

Any suitable route of administration may be employed for providing a human or non-human with the pharmaceutical composition of the invention. For example, oral, rectal, parenteral, sublingual, buccal, intravenous, intraarticular, intra-muscular, intra-dermal, subcutaneous, inhalational, intraocular, intraperitoneal, intracerebroventricular, transdermal and the like may be employed.

Preferably, the pharmaceutical composition of the invention is administered orally.

Pharmaceutical compositions of the present invention suitable for administration may be presented in discrete units such as vials, capsules, sachets or tablets each containing a predetermined amount of one or more pharmaceutically active compounds of the invention, as a powder or granules or as a solution or a suspension in an aqueous liquid, a non- aqueous liquid, an oil-in-water emulsion or a water-in-oil emulsion. Such compositions may be prepared by any of the methods of pharmacy but all methods include the step of bringing into association one or more pharmaceutically active compounds of the invention with the carrier which constitutes one or more necessary ingredients.

In general, the compositions are prepared by uniformly and intimately admixing the agents of the invention with liquid carriers or finely divided solid carriers or both, and then, if necessary, shaping the product to the desired presentation. In powders, the carrier is a finely divided solid which is in a mixture with the finely-divided active component.

In tablets, the active component is mixed with the carrier having the necessary binding capacity in suitable proportions and compacted in the shape and size desired.

The powders and tablets may contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

Tablets, powders, capsules, pills, cachets, and lozenges can be used as solid forms suitable for oral administration.

Liquid form preparations include solutions, suspensions, and emulsions, for example, water or water-propylene glycol solutions. For example, parenteral injection liquid preparations can be formulated as solutions in aqueous polyethylene glycol solution. The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative.

The compositions may take such forms as suspensions, solutions, or emulsions in oily or aqueous vehicles, and may contain formulatory agents such as suspending, stabilising and/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use. Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations-which are intended to be converted, shortly before use, to liquid form preparations for oral administration. Such liquid forms include solutions, suspensions, and emulsions. These preparations may contain, in addition to the active component, colorants, flavours, stabilizers, buffers, artificial and natural sweeteners, dispersants, thickeners, solubilising agents, and the like.

A ninth aspect of the invention provides a nutritional supplement comprising an effective amount of a compound of the first, second, third and/or fifth aspect, or a pharmaceutically acceptable salt thereof, and an additive.

The nutritional supplement may be prepared in an ingestible solid form such as capsules, tablets, powders, pills or granules. In the solid form, the additive may be fillers, binders and humectants. Further additives may include excipients and/or processing aides and/or vitamins and minerals. Exemplary excipients and processing aids, include but are not limited to, absorbents, diluents, flavorants, colorants, stabilizers, fillers, binders, disintegrants, lubricants, glidants, antiadherents, sugar or film coating agents, buffers, artificial sweeteners, natural sweeteners, dispersants, thickeners, solubilizing agents and the like or some combination thereof.

The supplements may also be prepared as a liquid solution, suspension or dispersion. Liquid forms include carriers such as water and ethanol, with or without other additives such as pharmaceutically acceptable surfactants or suspending agents.

Preferably, the compound of the first, second, third and/or fifth aspect in the nutritional supplement is selected from the group consisting of tricin-4′-O-[erythro-β-guaiacyl-(9″-O-p-coumaroyl)-glyceryl]ether (compound 7), tricin-4′-O-[threo-β-guaiacyl-(9″-O-p-coumaroyl)-glyceryl]ether (compound 8), tricin-4′-O-[threo-β-guaiacyl-(7″-O-methyl)-glyceryl]ether (compound 5) and tricin-4′-O-[erythro-β-guaiacyl-(7″-O-methyl)-glyceryl]ether (compound 6).

A tenth aspect of the invention provides for the use of a compound of the first, second, third and/or fifth aspect, or a pharmaceutically acceptable salt thereof, in the manufacture of a medicament for the treatment or prophylaxis of a disease, disorder or condition.

The compound may be administered orally to a patient and may be compounded in the form of syrup, tablets or capsule. When in the form of a tablet, any pharmaceutical carrier suitable for formulating such solid compositions may be used, for example magnesium stearate, starch, lactose, glucose, rice, flour and chalk. The compound may also be in the form of an ingestible capsule comprising, for example, gelatin to contain the compound, or in the form of a syrup, a solution or a suspension. Suitable liquid pharmaceutical carriers include ethyl alcohol, glycerine, saline and water to which flavouring or colouring agents may be added to form syrups. Sustained release formulations, for example tablets containing an enteric coating, are also envisaged. Various formulations and dosage forms have already been discussed herein.

The present invention further contemplates a combination of therapies, such as the administration of a compound of the first, second, third and/or fifth aspect, or a pharmaceutically acceptable salt thereof, together with the exposure of the subject to other agents or procedures which are useful in the treatment and/or control of postprandial hyperglycemia and its related conditions. The compounds of the invention may be administered simultaneously, separately or sequentially with the other agents or procedures.

An “effective amount” means an amount necessary at least partly to attain the desired response, or to delay the onset or inhibit progression or halt altogether, the onset or progression of a particular condition being treated. The amount varies depending upon the health and physical condition of the individual to be treated, the taxonomic group of individual to be treated, the degree of protection desired, the formulation of the composition, the assessment of the medical situation, and other relevant factors. It is expected that the amount will fall in a relatively broad range that can be determined through routine trials. An effective amount in relation to a human patient, for example, may lie in the range of about 0.1 ng per kg of body weight to 1 g per kg of body weight per dosage. The dosage may be in the-range of 1 μg to 1 g per kg of body weight per dosage, such as is in the range of 1 mg to 1 g per kg of body weight per dosage. In one embodiment, the dosage is in the range of 1 mg to 500 mg per kg of body weight per dosage. In another embodiment, the dosage is in the range of 1 mg to 250 mg per kg of body weight per dosage, In yet another embodiment, the dosage is in the range of 1 mg to 100 mg per kg of body weight per dosage, such as up to 50 mg per kg of body weight per dosage, in yet another embodiment, the dosage is in the range of 1 μg to 1 mg per kg of body weight per dosage.

Dosage regimes may be adjusted to provide the optimum therapeutic response. For example, several divided doses may be administered daily, weekly, monthly or other suitable time intervals, or the dose may be proportionally reduced as indicated by the exigencies of the situation.

References herein to “treatment” and “control” are to be considered in their broadest context. The term “treatment” does not necessarily imply that a subject is treated until total recovery. Similarly, “control” does not mean the subject experiences no effects of the disease or condition and does not necessarily mean that the subject will not eventually contract a disease condition. Accordingly, treatment and control include amelioration of the symptoms of a particular condition and preventing or otherwise reducing the effects of the condition or risk of developing a particular condition as well as reducing the severity or onset of a particular condition. “Treatment” and “control” may also reduce the severity of an existing condition.

So that the invention may be readily understood and put into practical effect, the following non-limiting examples are provided.

### Experimental

### Isolation of Compounds

Sugarcane leaves (Q136) were collected from a cane farm in Ballina (NSW, Australia) on three separate occasions. The leaf material was dried at 40° C., and ground using an industrial cutting mill (Retsch, SM2000). Dried and ground sugar cane leaves (13 kg) were sequentially extracted for 48 hours, using a wall-mounted rocker bin with dichloromethane (3×20 L) and methanol (3×20 L)as the extraction solvents. The methanolic extracts were individually concentrated by rotary evaporation, but were later combined as they had similar HPLC profiles. The methanolic sugarcane leaf extract (1.5 L) was suspended in water (10 L), centrifuged at 472 g for 10 minutes, and the water-soluble components were decanted to give an aqueous fraction (491 g) and an organic residue (94.5 g).

The organic residue was dissolved in a minimal amount of solvent (25% acetonitrile in water) before passing the solution over a column packed with C₁₈ stationary phase (prepared as described by O'Neill [1]). The separation of metabolites was achieved by step-wise gradient elution (2 bed volumes) under vacuum, eluting with 25%, 50%, 75% and 100% acetonitrile/water mixtures). The 50% fraction was evaporated to dryness using a rotary vacuum concentrator (RVC) (Christ, Germany) and redissolved in a minimal volume of 40% acetonitrile: water. The 50% fraction was applied to a second column containing a C₁₈ stationary phase (as described previously), and eluted with 30%, 40%, 50% and 100% acetonitrile/water mixtures.

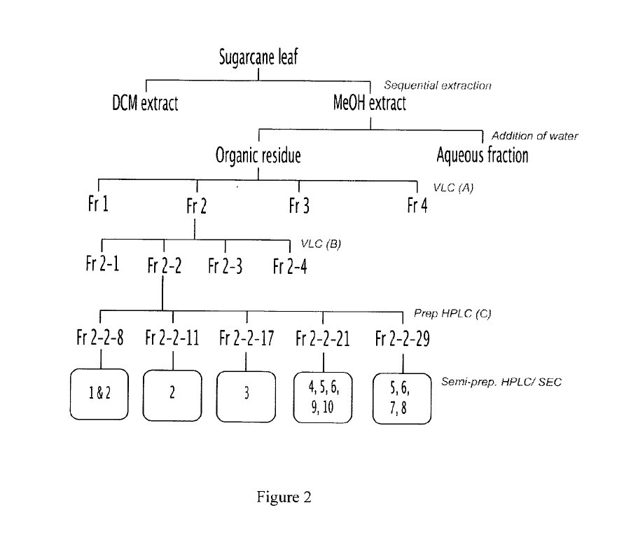

The 40% fraction was evaporated to dryness by RVC, and redissolved in 40:60 methanol and water. The resulting solution was subjected to preparative HPLC using 0.05% trifluoroacetic acid in water (Solvent A) and 0.05% trifluoroacetic acid in methanol (Solvent B), as eluents. Forty fractions were obtained over a 40-60% gradient of solvent B, run over a 45 minute period. Five fractions were further subjected to semi-preparative HPLC and size-exclusion chromatography to afford a number of compounds. FIG. 2 schematically represents this iterative isolation process.

In FIG. 2 the term VLC (vacuum liquid chromatography) refers to a process of eluting with (A) 25, 50, 75 & 100% MeCN/H₂O and (B) 30, 40, 50, 100% MeCN/H₂O. The preparative HPLC process incorporated eluting with (C) 40-60% MeOH/H₂O gradient. A Gilson preparative HPLC system with a binary pump (306) was used with a dual wavelength UV/VIS detector and a Phenomenex Luna C₁₈ (5μ, 150×22 mm i.d.) column. A Gilson FC 204 fraction collector was employed.

The preparative HPLC was carried out using eluting solution A (Milli-Q water and 0.05% TFA) and eluting solution B (methanol and 0.05% TFA) at a flow rate of 15 mL/min with one minute fractions collected. The eluting gradient used is that shown below.

| TIME (MIN) | % A | % B |
|---|---|---|
| 0 | 60 | 40 |
| 45 | 40 | 60 |
| 47 | 5 | 95 |
| 52 | 5 | 95 |
| 54 | 60 | 40 |
| 60 | 60 | 40 |

Semi-preparative HPLC was carried out eluting with isocratic MeCN/TFA/H₂O solvent mixtures (between 30 to 45% MeCN depending on analyte—detailed below) and size exclusion chromatography (SEC) eluting with 50% CHCl₃/MeOH. The semi-preparative HPLC process incorporated eluting with (C) 40-60% MeOH/H₂O gradient. An Agilent 1100 system with a quaternary pump was used with a diode array detector (DAD) and a Phenomenex Luna C₁₈ (5μ, 250×10 mm i.d.) column. A Gilson FC 204 fraction collector was employed.

The semi-preparative HPLC was carried out using eluting solution A (Milli-Q water) and eluting solution B (acetonitrile) at a flow rate of 1 mL/min with one minute fractions collected. The eluting solvent, fraction identification number and the compounds found within each fraction are shown below.

| FRACTION | SOLVENT | COMPOUND |
|---|---|---|
| Fr 2-2-21 | 45% Solvent B | 4, 5 and 6, |
| Fr 2-2-29 | 45% Solvent B | 5, 6, 7, 8, |

### Identification of Compounds 7 and 8

Tricin-4′-O-[erythro-β-guaiacyl-(9”-O-p-coumaroyl)-glyceryl]ether (compound 7) was isolated in a weight of 2.4 mg as an amorphous pale yellow solid. Spectral data is as follows: UV λₘₐₓ nm (CH₃CN): 273 sh, 290 sh, 313; APCl m/z: 673 [M+H]⁺; ¹H and ¹³C NMR (500 and 125 MHz, respectively, CD₃OD) are shown in table 2. Table 2 is a comparison of ¹H and ¹³C NMR spectral data for compounds 7 and 8 with literature values (in CD₃OD). In table 2 the literature data being used as a comparison is taken from Nakajima et al (reference [2]) and the superscripts a, b, c and d refer to assignments that could have been interchanged within that data.

Tricin-4′-O-[threo-β-guaiacyl-(9″-O-p-coumaroyl)-glyceryl]ether (compound 8) was isolated in the amount of 3.0 mg as an amorphous pale yellow solid. Spectral data is as follows: UV λₘₐₓ nm (CH₃CN): 273 sh, 290 sh, 313; APCl m/z: 673 [M+H]⁺; ¹H and ¹³C NMR (500 and 125 MHz, respectively, CD₃OD) are shown in Table 2.

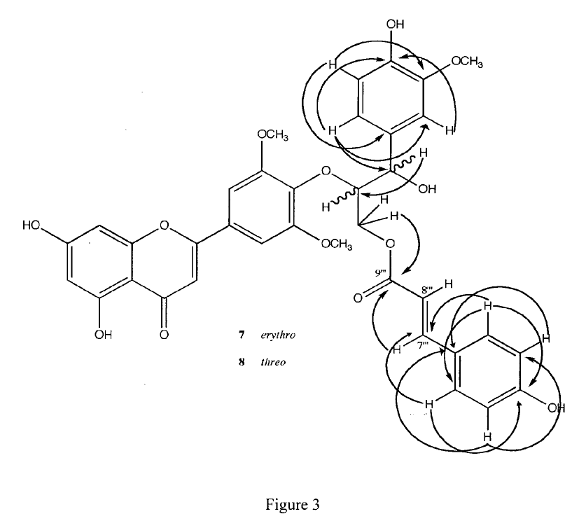

As mentioned above, the structures of compounds 7 and 8 were identified by spectroscopic data (¹H NMR, ¹³C NMR, and MS). The NMR assignments for compounds 7 and 8 were compared with those reported in the literature [2], which were obtained using deuterated methanol (CD₃OD) as the solvent. However, the published assignments for C-3″, C-5″, H-2′″, H-6′″, H-3′″ and H-5′″ differed from those obtained and displayed in table 2. The correct assignments were confirmed by the long-range J_{CH} correlations, as illustrated in FIG. 3, in addition to comparison of known chemical shift data for tricin-4′-O-(erythro-β-guaiacylglyceryl)ether, tricin-4′-O-(threo-β-guaiacylglyceryl)ether [3], and the cinnamic moiety of tricin-7-O-β-(6″-methoxycinnamic)-glucoside [4].

Identification of Compounds 5 and 6

Tricin-4′-O-[threo-β-guaicyl-(7″-O-methyl)-glyceryl]ether (compound 5) was isolated in the amount of 6.2 mg as yellow crystals. Spectral data is as follows: UV λₘₐₓ nm (CH₃OH): 280, 235sh, 205; APCl m/z: 541 [M+H]⁺; ¹H and ¹³C NMR (500 and 125 MHz, respectively, CD₃OD) are shown in Table 3.

Tricin-4′-O-[erythro-β-guaicyl-(7″-O-methyl)glyceryl]ether (compound 6) was isolated in the amount of 7.3 mg as an amorphous yellow solid. Spectral data is as follows; UV λₘₐₓ nm (CH₃OH): 280, 235sh, 205; APCl m/z: 541 [M+H]⁺; ¹H and ¹³C NMR (500 and 125 MHz, respectively, CD₃OD) are shown in Table 3.

Compound 5 was obtained as yellow crystals, while compound 6 was obtained as an amorphous yellow powder. The MS spectrum for both compounds 5 and 6 showed a molecular ion [M +H] at m/z 541, consistent with a molecular formula of C₂₈H₂₈O₁₁. The presence of a fragment ion at m/z 331, suggested the presence of a tricin moiety. ¹³C NMR and HMBC spectral data of 5 and 6 showed 13 quaternary carbons, ten methines, one methylene and four methyl groups. Examination of the ¹H NMR spectra of 5 showed the presence of eight aromatic protons. The singlet at 7.25 ppm suggested the presence of two identical aromatic protons (H-2′ and H-6′), while the doublets at 6.23 and 6.50 ppm gave a ³J_{HH} value of 2.1 Hz indicating meta coupling of H-6 and H-8, respectively.

The presence of a singlet at 6.72 ppm (H-3) as well as the two equivalent methoxy groups at 3.94 ppm, confirmed the structure of the tricin aglycone. The three remaining aromatic protons at 6.96, 6.83 and 6.80 ppm, were distributed over an ABD ring system and were assigned to H-2″, H-5″ and H-6″, respectively. The ³J_{CH} correlations from H-2″ and H-6″ to C-4″ (δ 147.5 ppm) and from H-5″ to C-3″ (8 149.0 ppm) established the position of the oxygenated quaternary carbons, and showed the presence of a methyl group (δ 56.6 ppm) attached via the oxygen substituent of C-3″. Two oxymethine protons were observed at 4.54 and 4.45 ppm (H-7″ and H-8″, respectively) along with two oxymethylene protons resonating at 3.63 and 3.34 ppm (H-9a″ and H-9b″). COSY and HMBC showed that these protons were arranged as a glycerol moiety and ²J_{CH} correlation of H-7″ to C-1″ showed how it was connected to the aromatic ring. The ₃J_{CH} correlation between the methoxy protons at 3.18 ppm and C-7″ (δ 85.5 ppm) allowed the final methoxy group to be placed at the C-7″ position.

The resonances and splitting patterns seen in the ¹H and ¹³C spectral data for compound 5 were similar to those seen in compound 6. However, the differences in chemical shifts and coupling constants suggested that 5 and 6 were diastereoisomers, which differed at the adjacent chiral centres at C-7″ and C-8″. The ³J_{H-7″, H-8″} coupling constants of 4′-O-8″ neolignans are known to be smaller in the erythro compared to the threo forms with greater variation seen for the threo (5.0-8.2 Hz) compared to the erythro (4.5-5.4 Hz) form when run in different solvents. The ³J_{HH} coupling constant in deuterated methanol was 6.7 Hz for compound 5, but was not determined for compound 6 due to overlapping proton signals of H-7″ and H-8″. Measurement of the coupling constants for compounds 5 and 6 in deuterated chloroform gave ³J_{HH} values of 7.7 and 6.1 Hz, respectively. Thus, the structure of compound 5 was determined as tricin-4′-O-[threo-β-guaicyl-(7″-O-methyl)glyceryl]ether and that of compound 6 as tricin-4′-O-[erythro-β-guaicyl-(7″-O-methyl)-glyceryl]ether.

In vitro Enzyme Assays

Bakers yeast α-glucosidase [EC 3.2.1.20], and porcine pancreatic α-amylase [EC 3.2.1.1]were used during bioassay guided fractionation. Rat intestinal acetone powder was used as a source of mammalian α-glucosidase. The EnzChek® Ultra Amylase Assay kit (E-33651) was purchased from Molecular Probes (Eugene, Oreg., USA). Acarbose (Glucobay, 50 mg/tablet) was obtained from a local pharmacy. The reference standards apigenin and epigallocatechin gallate were purchased from Chromadex (Santa Ana, Calif., USA).

In each bioassay the hydrolysis product was measured using the Victor 2 multi-plate reader (Wallac, Turku, Finland) and all samples were tested in duplicate or triplicate. Non-enzyme controls were subtracted from the enzyme absorbance and the percentage inhibition of test samples was calculated as:

((A−B)/A)×100

:wherein A and B are the absorbance of the hydrolysis product in the absence and presence of inhibitor, respectively. Percent inhibition values were expressed as mean±standard deviation. The IC₅₀ was the concentration required for 50% inhibition of enzyme activity under the assay conditions, and values were calculated using a four-parameter fit by Microsoft Excel Solver.

Compounds 4, 18 and 19, as listed in table 1, were chosen as reference standards for inclusion in the assays as they had previously been isolated from sugarcane or sugar manufacturing products. Apigenin was purchased from Chromdex (Santa Ana, Calif., USA) while Luteolin was purchased from the Sigma-Aldrich Chemical Company (Castle Hill, Australia).

### Yeast α-Glucosidase Inhibition Assay

Extracts and fractions were dissolved in DMSO at 30 mg/mL or 10 mM for pure compounds, and diluted to working concentrations in sodium acetate buffer (pH 5.5). Final sample concentrations were 50 μg/mL, unless otherwise stated. The substrate, 4-methylumbelliferyl-α-D-glucopyranoside (84 μM, 45 μL), was added to 96-well plates containing 50 μL yeast α-glucosidase (3 mU/mL) and 5 μL of sample. The plate was mixed on an orbital shaker for 30 seconds and incubated for 20 minutes at 37° C. The reaction was stopped by the addition of 100 mM sodium-glycine buffer (100 μL, pH 10.6) and the plate was shaken for a further 30 seconds and the fluorescence intensity was measured at λₑₓ 355 nm, λₑₘ 460 nm. Fucoidan (20 μg/mL) was used as a positive inhibitor control, and sodium acetate buffer as a negative control.

### Mammalian α-Glucosidase Inhibition Assay

The α-glucosidase inhibitory activity of pure compounds was measured as described previously [5], with some modifications. The crude enzyme solution was prepared by suspending 50 mg of rat intestinal acetone powder in 0.9% sodium chloride solution (1.5 mL). The mixture was homogenised by sonication in ice-cold water for 10 minutes and then centrifuged at 10,000 g for 30 minutes. The resulting supernatant was used in the assay.

Pure compounds were dissolved in DMSO and diluted in 100 mM sodium phosphate buffer (pH 6.8) to give final concentrations of 100 μM. The substrate maltose (3 mM, 20 μL) was added to 96-well plates containing the crude α-glucosidase mixture (20 μL) and sample (10 μL). The microtitre plate was mixed on an orbital shaker for 30 seconds, incubated for 30 minutes at 37° C. and the reaction was stopped by the addition of 2 M Tris buffer (75 μL ). The glucose liberated was measured by adding 30 μL of the reaction mixture to the Glucose Hexokinase assay reagent (170 μL), which was mixed and incubated for 15 minutes at room temperature. The fluorescence of the mixture was measured at λₑₓ 340 nm, λₑₘ 470 nm. Acarbose (30 μM) was used as a positive control, and sodium phosphate buffer as a negative control.

### Porcine α-Amylase Inhibition Assay

This assay was performed using the EnzChek® Ultra Amylase Assay kit. Samples were dissolved in DMSO and diluted with 100 mM of 3-morpholinopropanesulfonic acid (MOPS) buffer (pH 6.9) to give a final concentration of 300 pg/mL, unless otherwise stated. The substrate (95 μL), a starch compound labelled with a fluorescent (BODIPY) dye, was added to microtitre plates containing 95 μL α-amylase solution (125 U/mL) and 10 μL of sample. The fluorescence intensity was continuously measured at λₑₓ 485 nm, λₑₘ 538 nm for 15 minutes. Acarbose was used as a positive control, and MOPS buffer (pH 6.9) was used as a negative control.

The results of the three assays are displayed in table 1 and have already been discussed herein.

The present invention provides for compounds of formulae I to V which are useful as GI lowering agents. The invention also includes nutritional and/or pharmaceutical compositions/supplements comprising one or more of these compounds. The compounds will be beneficial to patients who require stabilization of their postprandial glucose levels by inhibition of the main carbohydrate digestive enzymes, α-amylase and α-glucosidase. Inhibition of one or both of these enzymes results in a delay in the digestion of carbohydrates and hence delays the production and subsequent absorption of glucose into the bloodstream. This enables postprandial blood glucose levels to be reduced.

Such control is useful, for example, to discourage over-eating in patients suffering from obesity by extending the time frame of the feeling of satiety or otherwise trying to reduce calorie intake or in patients suffering from, or with a pre-disposition to, diabetes and related conditions.

The compounds of the invention may be administered to a patient as a single isolated and purified compound, which may be delivered within a nutritional or pharmaceutical composition, or as a sugarcane extract comprising a plurality of such compounds.

It will be appreciated by the skilled person that the present invention is not limited to the embodiments described in detail herein, and that a variety of other embodiments may be contemplated which are, nevertheless, consistent with the broad spirit and scope of the invention.

All computer programs, algorithms, patent and scientific literature referred to in this specification are incorporated herein by reference in their entirety.

### REFERENCES

[1] I. A. O'Neill, Reverse Phase Flash Chromatography: A Convenient Method for the Large Scale Separation of Polar Compounds, Synlett (1991) 661-662.
[2] Y. Nakajima, Y. S. Yun, and A. Kunugi, Six New Flavonolignans from Sasa veitchii (Carr.) Rehder, Tetrahedron 59 (2003) 8011-8015.
[3] M. Bouaziz, N. C. Veitch, R. J. Grayer, M. S. J. Simmonds, and M. Damak, Flavonolignans from Hyparrhenia hirta, Phytochemistry 60 (2002) 515-520.
[4] J. M. Duarte-Almeida, G. Negri, A. Salatino, J. E. de Carvalho, and F. M. Lajolo, Antiproliferative and Antioxidant Activities of a Tricin Acylated Glycoside from Sugarcane (Saccharum officinarum) Juice, Phytochemistry 68 (2007) 1165-1171.
[5] T. Oki, T. Matsui, and Y. Osajima, Inhibitory Effect of Alpha-Glucosidase Inhibitors Varies According to Its Origin, Journal of Agricultural and Food Chemistry 47 (1999) 550-553.

### Tables

TABLE 1
Inhibitory activity of compounds isolated from methanolic sugarcane
extract and controls

| | | | | Glucosidase |
|---|---|---|---|---|
| | | Amylase | Glucosidase | (rat) |
| | | (porcine) | (yeast) | % Inhibition |
| CMP | Name | IC₅₀ (μM) | | at 200 μM |
| 4 | Tricin | 104.2 | 70.6 | 16.1 |
| 5 | Tricin-4′-O-[threo-β- | 31.9 | 95.9 | 6.1 |
| | guaiacyl-(7″-O- | | | |
| | methyl)-glyceryl] ether | | | |
| 6 | Tricin-4′-O-[erythro- | 26.5 | 72.0 | 6.1 |
| | β-guaiacyl-(7″-O- | | | |
| | methyl)-glyceryl] ether | | | |
| 7 | Tricin-4′-O-[erythro- | 2.0 | 37.5 | 25.7 |
| | β-guaiacyl-(9″-O-p- | | | |
| | coumaroyl)-glyceryl] | | | |
| | ether | | | |
| 8 | Tricin-4′-O-[threo- | 0.9 | 37.9 | 10.0 |
| | β-guaiacyl-(9″-O-p- | | | |
| | coumaroyl)-glyceryl] | | | |
| | ether | | | |
| 18 | Apigenin | 189.6 | 108.4 | −12.2 |
| 19 | Luteolin | 99.5 | 92.3 | 4.8 |
| | Acarbose | 121.4 | 225.1 | Approx. |
| | | | | 100%* |
| | Fucoidan (μg/mL) | — | 0.82 | — |

*N.b. Acarbose inhibition would be near 100% at lower concentrations of between 100 to 150 μM.

TABLE 2

| | 7 | | Literature Values | | 8 | | Literature Values | |
|---|---|---|---|---|---|---|---|---|
| POSITION | ¹H | ¹³C | ¹H | ¹³C | ¹H | ¹³C | ¹H | ¹³C |
| 2 | | 165.0 | | 165.2 | | 165.0 | | 165.0 |
| 3 | 6.60 s | 105.5 | 6.60 s | 105.6 | 6.62 s | 105.9 | 6.57 s | 105.4 |
| 4 | | 183.5 | | 183.9 | | 183.9 | | 183.7 |
| 5 | | 163.0 | | 163.4 | | 163.0 | | 163.2 |
| 6 | 6.20 d(2.2) | 101.0 | 6.21 d(1.9) | 100.6 | 6.22 d(2.1) | 100.6 | 6.20 d(1.9) | 100.4 |
| 7 | | 166.5 | | 166.7 | | 166.5 | | 166.5 |
| 8 | 6.38 d(2.2) | 95.5 | 6.39 d(1.9) | 95.4 | 6.40 d(2.1) | 95.4 | 6.37 d(1.9) | 95.2 |
| 9 | | 159.6 | | 159.6 | | 159.6 | | 159.4 |
| 10 | | 105.5 | | 105.9 | | 105.9 | | 105.7 |
| 1′ | | 127.7 | | 127.0 | | 128.0 | | 126.7 |
| 2′,6′ | 7.24 s | 105.0 | 7.20 s | 105.1 | 7.21 s | 105.3 | 7.16 s | 105.0 |
| 3′,5′ | | 154.7 | | 155.0 | | 155.0 | | 154.8 |
| 3′,5′-OCH₃ | 3.90 s | 57.0 | 3.90 s | 57.1 | 3.91 s | 57.1 | 3.88 s | 56.9 |
| 4′ | | 141.6 | | 141.6 | | 141.6 | | 141.4 |
| 1″ | | 133.5 | | 133.9 | | 133.4 | | 133.2 |
| 2″ | 7.11 d(1.8) | 111.5 | 7.10 d(1.7) | 111.7 | 7.09 d(1.9) | 112.1 | 7.09 d(1.8) | 111.8 |
| 3″ | | 149.0 | | 147.3^{b} | | 148.7 | | 147.4^{d} |
| 3″-OCH₃ | 3.88 s | 56.5 | 3.88 s | 56.6 | 3.86 s | 56.6 | 3.86 s | 56.4 |
| 4″ | | 147.2 | | 149.0^{b} | | 147.6 | | 148.8^{d} |
| 5″ | 6.80 d(8.2) | 116.0 | 6.80 d(8.1) | 116.0 | 6.80 d(8.1) | 116.1 | 6.80 d(8.1) | 115.9 |
| 6″ | 6.92 dd(8.2, 1.8) | 121.0 | 6.90 dd(8.1, 1.7) | 120.8 | 6.93 dd(8.1, 1.9) | 121.1 | 6.93 dd(8.1, 1.8) | 120.9 |
| 7″ | 5.01 d(4.9) | 75.0 | 5.00 d(4.9) | 74.8 | 4.98 d(6.0) | 75.2 | 4.98 d(6.0) | 75.1 |
| 7″-OCH₃ | | | | | | | | |
| 8″ | 4.76 m | 84.7 | 4.81 m | 84.8 | 4.77 m | 85.2 | 4.74 m | 85.1 |
| 9a″ | 4.57 dd(11.9, 7.8) | 65.6 | 4.55 dd(11.9, 7.7) | 66.0 | 4.24† | 66.6 | 4.22 dd(11.9, 6.4) | 66.5 |
| 9b″ | 4.38 dd(11.9, 2.4) | | 4.36 dd(11.9, 2.3) | | 4.24† | | 4.24 dd(11.9, 2.8) | |
| 1″′ | | 126.5 | | 127.8 | | 127.0 | | 127.7 |
| 2″′,6″′ | 7.24 d(8.6) | 131.2 | 6.60 d(8.6)^{a} | 131.2 | 7.25 d(8.7) | 131.2 | 6.61 d(8.6)^{c} | 131.2 |
| 3″′,6″′ | 6.62 d(8.6) | 116.5 | 7.23 d(8.6)^{a} | 116.9 | 6.62 d(8.6) | 116.9 | 7.25 d(8.6)^{c} | 116.7 |
| 4″′ | | 161.2 | | 161.4 | | 161.6 | | 161.3 |
| 7″′ | 7.06 d(16.0) | 146.5 | 7.04 d(16.0) | 146.5 | 7.10 d(16.0) | 146.7 | 7.09 d(15.9) | 146.5 |
| 8″′ | 6.02 d(16.0) | 114.9 | 6.01 d(16.0) | 114.9 | 6.04 d(16.0) | 114.7 | 6.03 d(15.9) | 114.5 |
| 9″′ | | 168.8 | | 168.9 | | 168.5 | | 168.6 |

^{a,b,c,d}Values with the same superscript are interchangeable

TABLE 3
Table 3: NMR spectral data for compounds 5 and 6

| | COMPOUND 5 | | COMPOUND 6 | |
|---|---|---|---|---|
| POSITION | ¹H | ¹³C | ¹H | ¹³C |
| 2 | | 165.7 | | 165.5 |
| 3 | 6.72 s | 105.9 | 6.66 s | 106.0 |
| 4 | | 184.0 | | 184.0 |
| 5 | | 163.3 | | 162.9 |
| 6 | 6.23 d (2.0) | 100.4 | 6.21 d (1.9) | 100.4 |
| 7 | | 166.5 | | 166.5 |
| 8 | 6.50 d (2.0) | 95.4 | 6.46 d (1.9) | 95.3 |
| 9 | | 159.7 | | 159.6 |
| 10 | | 105.9 | | 105.7 |
| 1′ | | 127.6 | | 127.8 |
| 2′, 6′ | 7.26 s | 105.4 | 7.16 s | 105.3 |
| 3′, 5′ | | 155.0 | | 154.9 |
| 3′, 5′-OCH₃ | 3.94 s | 57.1 | 3.87 s | 57.0 |
| 4′ | | 141.5 | | 140.9 |
| 1″ | | 131.2 | | 131.3 |
| 2″ | 6.96 d (1.7) | 112.4 | 6.90 d (1.7) | 112.5 |
| 3″ | | 149.0 | | 149.0 |
| 3″-OCH₃ | 3.85 s | 56.6 | 3.81 s | 56.6 |
| 4″ | | 147.5 | | 147.6 |
| 5″ | 6.80 d (8.1) | 116.2 | 6.75 d (8.1) | 115.8 |
| 6″ | 6.83 dd (8.1, 1.7) | 121.8 | 6.78 dd (8.1, 1.7) | 122.3 |
| 7″ | 4.54 d (6.7) | 85.5 | 4.46 | 84.1 |
| 7″-OCH₃ | 3.18 s | 57.3 | 3.25 s | 57.3 |
| 8″ | 4.45 m | 87.0 | 4.46 m | 86.8 |
| 9a″ | 3.63 dd (11.9, | | 3.94 dd (11.9, | |
| | 3.8) | | 4.0) | |
| 9b″ | 3.35 dd (11.9, | 62.6 | 3.76 dd (11.9, | 63.1 |
| | 1.7) | | 2.2) | |

1. A compound of formula I, and/or a salt thereof, for use as a glycemic index lowering agent and/or, as an α-amylase and/or α-glucosidase inhibitor: 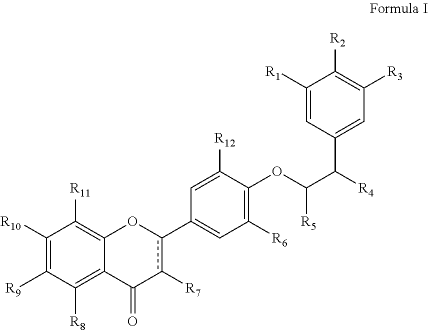
wherein,
R₁, R₂, R₃, R₄, R₆, R₇, R₈, R₉, R₁₀, R₁₁ and R₁₂ are independently selected from hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety;
R₅ is hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl, a sugar moiety or R₅ may be represented by the following structure: 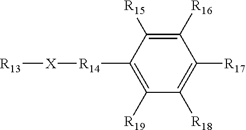
wherein, R₁₃ and R₁₄ are independently selected from alkyl, aryl, alkylene, alkenyl, alkynyl, alkanone, alkanoyl, arylalkyl, arylalkenyl, alkenoyl or carboalkoxy;
X, when present, is oxygen, sulphur, nitrogen, alkyl, alkoxy, alkanoyloxy, alkylene or alkenyl;
R₁₅, R₁₆, R₁₇, R₁₈ and R₁₉ are independently selected from hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety; and
wherein dotted lines may each represent a single bond.

2-5. (canceled)

6. The compound of claim 1 wherein the compound is selected from the group consisting of tricin-4′-O-[threo-β-guaiacyl-(9″-O-p-coumaroyl)-glyceryl]ether, tricin-4′-O-[erythro-β-guaiacyl-(9″-O-p-coumaroyl)-glyceryl]ether, tricin-4′-O-[threo-β-guaiacyl-(7″-O-methyl)-glyceryl]ether and tricin-4′-O-[erythro-β-guaiacyl-(7″-O-methyl)-glyceryl]ether.

7. A compound of formula I, and/or a salt thereof: 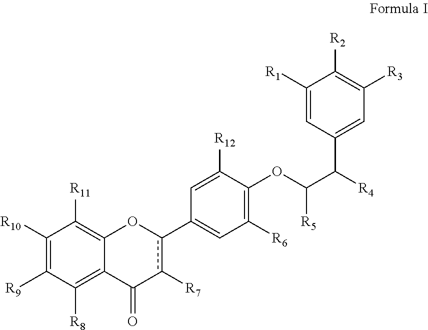
wherein,
R₁, R₂, R₃, R₄, R₆, R₇, R₈, R₉, R₁₀, R₁₁ and R₁₂ are independently selected from hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety;
R₅ is hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl, a sugar moiety or R₅ may be represented by the following structure: 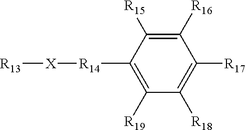
wherein, R₁₃ and R₁₄ are independently selected from alkyl, aryl, alkylene, alkenyl, alkynyl, alkanone, alkanoyl, arylalkyl, arylalkenyl, alkenoyl or carboalkoxy;
X, when present, is oxygen, sulphur, nitrogen, alkyl, alkoxy, alkanoyloxy, alkylene or alkenyl;
R₁₅, R₁₆, R₁₇, R₁₈ and R₁₉ are independently selected from hydrogen, alkyl, alkenyl, arylalkyl, hydroxyalkyl, hydroxyl, aldehyde, alkanone, carboxyl, carboxamide, alkanoyl, carboalkoxy, carboaryloxy, carbonate, O-alkyl, O-aryl, O-alkenyl, O-alkanoyl, O-alkenoyl or a sugar moiety;
wherein dotted lines may each represent a single bond; and
wherein the compound is not tricin-4′-O-[threo-β-guaiacyl-(9”-O-p-coumaroyl)-glyceryl]ether, tricin-4′-O-[erythro-β-guaiacyl-(9″-O-p-coumaroyl)-glyceryl]ether, tricin-4′-O-(erythro-β-guaiacylglyceryl)ether or tricin-4′-O-(threo-β-guaiacylglyceryl)ether.

8. The compound of claim 7 wherein the compound is tricin-4′-O-[threo-β-guaiacyl-(7″-O-methyl)-glyceryl]ether and/or tricin-4′-O-[erythro-β-guaiacyl-(7″-O-methyl)-glyceryl]ether.

9. A method of isolating a compound of formula I, including the step of extracting said compound from a plant, plant part or plant derivative of the family Poaceae/Gramineae.

10. The method of claim 9 wherein the plant is a species selected from the group consisting of Saccharum officinarum, Saccharum spontaneum, Sasa veitchii (Can.) Rehder, Hyparrhenia hirta (L.) Stapf, Salsola collina, Avena sativa L. and Lycopodium japonicum.

11. The method of claim 9 wherein the extract is obtained from the leaves and/or stem of the plant and/or from a sugarcane processing waste stream.

12. The method of claim 11 wherein the sugarcane processing waste stream includes molasses, sugar syrup, field trash, growing tips and mill mud.

13. The method of claim 9 wherein the compound is extracted from a sugarcane leaf using methanol.

14-16. (canceled)

17. A method of treating a disease, disorder or condition responsive to a flavonoid or flavonoid derivative, including the step of administering a compound of claim 1.

18. The method of claim 17 wherein the disease, disorder or condition to be treated is selected from the group consisting of obesity, diabetes and diabetes related conditions.

19. A nutritional composition comprising a compound of claim 1, or a pharmaceutically acceptable salt thereof, and a nutritional component.

20. The nutritional composition of claim 19 wherein the compound is selected from the group consisting of tricin-4′-O-[threo-β-guaiacyl-(9″-O-p-coumaroyl)-glyceryl]ether, tricin-4′-O-[erythro-β-guaiacyl-(9″-O-p-coumaroyl)-glyceryl]ether, tricin-4′-O-[threo-β-guaiacyl-(7″-O-methyl)-glyceryl]ether and tricin-4′-O-[erythro-β-guaiacyl-(7″-O-methyl)-glyceryl]ether.

21. The nutritional composition of claim 19 further comprising a food additive selected from the group consisting of molasses, poly phenols, kidney bean and kidney bean extracts including phaseolamin, a fibre additive and an acid.

22. A pharmaceutical composition for the treatment or prophylaxis of a disease, disorder or condition comprising an effective amount of a compound of claim 1, or a pharmaceutically acceptable salt thereof, and a pharmaceutically acceptable carrier, diluent and/or excipient

23. The pharmaceutical composition of claim 22 wherein the compound is selected from the group consisting of tricin-4′-O-[threo-β-guaiacyl-(9″-O-p-coumaroyl)-glyceryl]ether, tricin-4′-O-[erythro-β-guaiacyl-(9″-O-p-coumaroyl)-glyceryl]ether, tricin-4′-O-[threo-β-guaiacyl-(7″-O-methyl)-glyceryl]ether and tricin-4′-O-[erythro-β-guaiacyl-(7″-O-methyl)-glyceryl]ether.

24-26. (canceled)
